(19) United States
(12) Patent Application Publication
(10) Pub. No.: US 2025/0163214 A1
(43) Pub. Date: May 22, 2025

(54) ABSORBABLE COPOLYMER, AND PREPARATION METHOD AND USE THEREOF

(71) Applicant: Jiangsu Greatchina Sino-Tech Biomedical Materials Co., Ltd, Changzhou (CN)

(72) Inventors: Shihua Jiang, Changzhou (CN); Tianyao Zhang, Changzhou (CN); Dongliang Chen, Changzhou (CN); Chengdong Xiong, Changzhou (CN)

(21) Appl. No.: 19/034,854

(22) Filed: Jan. 23, 2025

(30) Foreign Application Priority Data
Aug. 1, 2024 (CN) 202411050136.3

(51) Int. Cl. C08G 63/64

(52) U.S. Cl. CPC C08G 63/64

(57) ABSTRACT

An absorbable copolymer, and a preparation method and use thereof are disclosed. Firstly lactide and trimethylene carbonate are polymerized to form a prepolymer, and then the prepolymer is copolymerized with glycolide to form a PGLT copolymer. The PGLT copolymer contains three chain segments of polymerized glycolide, polymerized lactide and polymerized trimethylene carbonate. The PGLT copolymer obtained by stepwise polymerization has high strength and good toughness. It is suitable for the preparation of medical instruments such as absorbable tissue closure clips, suture clips, suture anchors, anastomosis nails, etc.

### TECHNICAL FIELD

The present disclosure belongs to the technical field of medical instruments, and particularly relates to an absorbable copolymer and a preparation method and use thereof.

### BACKGROUND

A tissue closure clip is an instrument commonly used in minimally invasive endoscopic surgical procedures, which is mainly used for clamping on pipeline tissues, where the tissue closure clip with a V-shaped locking structure has the best use effect. The tissue closure clip can be divided into an absorbable closure clip and a nonabsorbable closure clip according to whether it can be degraded in vivo, where the nonabsorbable closure clip is made of metal or a nondegradable polymer material, which greatly limits the clinical application of the nonabsorbable closure clip since it cannot be absorbed by human body.

An absorbable closure clip (Absolok Extra, U.S. Pat. No. 4,424,810) developed by Johnson & Johnson, United States has a V-shaped locking structure and is made of absorbable poly(p-dioxanone) PPDO. This material has good flexibility, but has low strength, and thus has an insufficient clamping force. In use, it will deform and cause the clamped tissue to slip or jump from the clip. When there is an omentum tissue around the pipeline tissue in the body, it is difficult to penetrate the omentum tissue to achieve closure.

Among degradable and absorbable materials, PGA (polyglycolic acid or polyglycolide) has high mechanical strength, high rigidity and good crystallinity, but lacks toughness and has a quick degradation speed, so it is difficult to be used as a V-shaped clip with large stress. Therefore, blending is usually used for improving the toughness of degradable polymer materials, for example blending the PGA with a relatively softer materials such as poly(p-dioxanone) (PPDO), polytrimethylene carbonate (PTMC) and polycaprolactone (PCL) to improve the toughness of the materials. However, due to compatibility problems, the degradation speed of the composite material is accelerated, which cannot meet the requirements of closure clip materials.

Although the toughness of a degradable polymer material GA can be improved by copolymerization, the crystallinity of a GA chain segment will decrease after the formation of a random copolymer, which will lead to the decrease of mechanical strength and the degradation speed being faster than that of the GA polymer. Stepwise block copolymerization can also increase the toughness of the PGA material. That is, a tough monomer is polymerized into a prepolymer, and then the prepolymer is used as a soft segment, and then copolymerized with the GA monomer to form a hard segment. However, the material formed by such a polymerization manner is difficult to control, and it is only suitable for sutures when the material is soft. If the strength is improved by increasing the content of GA in the hard segment, in turn the degradation rate will increase.

In view of the above, it can be seen that toughening and modification of the PGA by either blending or copolymerization cannot solve the problems of mechanical properties and degradation rates at the same time. Therefore, it is a hot spot in the field of medical instrument manufacturing to study degradable and absorbable medical materials with good mechanical properties, good rigid strength and good degradation speeds.

### SUMMARY

In order to solve the aforementioned problems, the present disclosure provides an absorbable copolymer, and a preparation method and use thereof.

The present disclosure adopts the following technical solution: Disclosed is a method for preparing an absorbable copolymer, which is a PGLT copolymer formed by stepwise copolymerization of glycolide, lactide and trimethylene carbonate, where in the PGLT copolymer, a weight percentage of the glycolide is 60%-85%, a weight percentage of the lactide is 5%-25%, and a weight percentage of the trimethylene carbonate is 8%-25%, and in a process of stepwise copolymerization, the lactide and the trimethylene carbonate are firstly polymerized to form a prepolymer and then the prepolymer is copolymerized with the glycolide, and the PGLT copolymer contains three chain segments of polymerized glycolide, polymerized lactide and polymerized trimethylene carbonate, specifically including the following steps:

(1) a reactant A: forming a homopolymer or copolymer by polymerization of the lactide or of the lactide and the glycolide in the presence of an initiator and a catalyst;

(2) a reactant B: forming a homopolymer or copolymer by polymerization of the trimethylene carbonate or of the trimethylene carbonate and the glycolide in the presence of an initiator and a catalyst; and

(3) copolymerization: using the glycolide as a reactant C, melting and blending the reactant C, the reactant B and the reactant A evenly, and then polymerizing in an air-insulated reactor to form the PGLT copolymer.

Further, the method for preparing the absorbable copolymer includes:

(1) a reactant A: adding 0.01%-0.2% by weight of the initiator and 0.01%-0.1% by weight of the catalyst, respectively adding the lactide and the glycolide into a reactor under the protection of nitrogen or a vacuum condition, heating to 150° C.-180°C., maintaining at the reaction temperature for 6-15 hours, and conducting ring-opening polymerization to obtain the reactant A;

(2) a reactant B: adding 0.01%-0.2% by weight of the initiator and 0.01%-0.1% by weight of the catalyst, respectively adding the trimethylene carbonate and the glycolide into a reactor under the protection of nitrogen or a vacuum condition, heating to 150° C.-180° C., maintaining at the reaction temperature for 2-8 hours, and conducting ring-opening polymerization to obtain the reactant B;

(3) copolymerization: using the glycolide as the reactant C, melting and blending the reactant C, the reactant A and the reactant B evenly, and polymerizing at 200° C.-230° C. in an air-insulated reactor to form the PGLT copolymer containing the glycolide, the lactide and the trimethylene carbonate.

Further, the initiator is a monohydric, dihydric or polyhydric alcohol among fatty alcohols, and the catalyst is stannous octoate.

Further, the lactide is one or more of L-lactide, DL-lactide or D-lactide.

Further, in the reactant A, the weight percentage of the glycolide is 0-20%, and in the reactant B, the weight percentage of the glycolide is 0-20%.

In the present disclosure, the proportions of the glycolide, the lactide and the trimethylene carbonate in the PGLT copolymer is the best scheme obtained by designing and adjusting the mechanical strength, hardness (or toughness) and degradation time of the copolymer according to the material requirements of the absorbable closure clip. Too high content of the lactide in the PGLT copolymer is not good for mechanical properties. For example, stress relaxation may occur during the use of the closure clip, which may lead to the loosening of a clamped pipeline. Too low content of the lactide in the PGLT copolymer will affect the degradation performance of the copolymer. Too high content of the trimethylene carbonate in the PGLT copolymer will reduce the mechanical properties of the copolymer, and too low content of the trimethylene carbonate in the PGLT copolymer will reduce the flexibility of the copolymer. If the content of the glycolide is too high, the copolymer will degrade faster and its toughness will deteriorate, while if the content of the glycolide is too low, the mechanical properties of the copolymer will be reduced.

The prepared absorbable copolymer has an elastic modulus of 600 MPa-2,300 MPa, a tensile yield strength of greater than 40 MPa, and a tensile elongation at break of no less than 40%.

Further, the PGLT copolymer has an elastic modulus of 800 MPa-2,000 MPa, a tensile yield strength of greater than 50 MPa, and a tensile elongation at break of no less than 50%.

Further, the PGLT copolymer has an intrinsic viscosity of 0.8-2.5 dl/g determined at 25° C. in hexafluoroisopropanol with a concentration of 0.1 g/dl.

Further, the tensile strength of the PGLT copolymer is no less than 5 MPa after being soaked in a phosphate buffer solution at 37° C. for 14 days.

Disclosed is use of an absorbable copolymer, and the absorbable copolymer can be used for preparing tissue closure clips, suture clips, suture anchors, anastomosis nails, surgical sutures, puncture occluders or anal fistula occludes.

The PGLT copolymer in the present disclosure contains three reactants: various combinations of a reactant A, a reactant B and a reactant C including: A-C+B+C, A-C-B, A-B-C, C-A-B, C-A-C, C-B-C, C-A-B-C, etc. The structural formula of the A-B-C is as shown in the figure below:

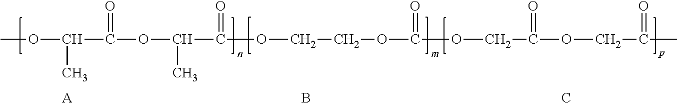

Compared with the random copolymer, the PGLT copolymer of the present disclosure fully displays the characteristics of each chain segment of the reactant A, the reactant B and the reactant C. Compared with the large amount of random polymerization between a GA monomer and a LA monomer in the existing polymer, in the present disclosure the lactide chain segment and the trimethylene carbonate chain segment that are formed by prepolymerization are copolymerized with glycolide by a stepwise copolymerization method, which is not greatly changed due to copolymerization, avoiding the problems that the crystallinity is not improved due to LA doped in the GA chain formed by a large amount of random copolymerization between the GA monomer and the LA monomer, and the copolymerized material has low strength and a fast degradation speed. The chain segment formed from the reactant C after polymerization with the glycolide has high crystallinity as a hard segment, which determines the high mechanical strength of the copolymer. The lactide chain segment formed from the reactant A has the characteristics of high mechanical strength and slow degradation as a hard segment, which mainly acts on the strength of the PGLT copolymer and slows down degradation. The trimethylene carbonate chain segment formed from the reactant B has the characteristics of flexibility and slow degradation as a soft segment, which makes the PGLT copolymer soft.

For the PGLT copolymer prepared by the present disclosure, the control of the intrinsic viscosity range of the copolymer can be realized through the intrinsic viscosity of the chain segments of the reactant A and the reactant B, where the intrinsic viscosity of the reactant A in chloroform with a concentration of 0.1 g/dl is 1.0-4.0 dl/g at 25° C.; and the intrinsic viscosity of the reactant B in chloroform with a concentration of 0.1 g/dl is 1.0-4.0 dl/g at 25° C.

The advantages of the present disclosure are as follows:

(1) The preparation method of the present disclosure is simple, and the steps of it are easy to operate. The copolymer is formed by the stepwise copolymerization method. Firstly, the lactide and the trimethylene carbonate are polymerized into a prepolymer, and then the prepolymer is copolymerized with the glycolide to form the copolymer, which greatly reduces the randomness in the copolymerization process;

(2) The copolymer prepared by the present disclosure is generated by copolymerization of a soft segment prepolymer-trimethylene carbonate and a copolymer thereof, a hard segment prepolymer-lactide and a copolymer thereof, and glycolide GA, and the copolymer retains the characteristics of each segment, which not only improves the toughness of the material, but also avoids the problem that the degradation speed is accelerated due to excessive content of the glycolide GA, and the mechanical strength, toughness and degradation time of the copolymer can be regulated by adjusting the ratio of each reactant, so as to prepare a polymer material suitable for absorbable medical instruments such as the absorbable closure clip, which has broad application prospects in the field of medical instruments.

### BRIEF DESCRIPTION OF THE DRAWINGS

FIG. 1 is a 1HNMR map of an absorbable copolymer of the present disclosure.

FIG. 2 is a DSC thermoanalysis diagram of the absorbable copolymer of the present disclosure.

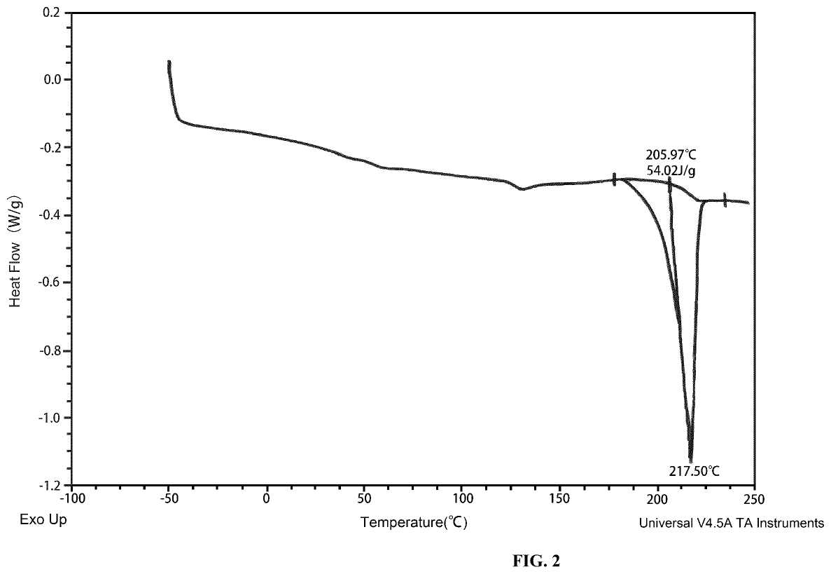

FIG. 3 is a schematic structural view of a V-shaped absorbable closure clip.

### DETAILED DESCRIPTION OF THE EMBODIMENTS

In order to make the aforementioned objectives, features and advantages of the present disclosure more obvious and easy to understand, the present disclosure will be further explained and illustrated in conjunction with specific examples hereafter.

### Example 1

The preparation of a PGLT copolymer 1 (PGLT1) of glycolide, L-lactide and trimethylene carbonate included the following steps:

(1) preparation of a reactant A: 200 g of a L-lactide monomer (LA) was placed in a 3 L stainless steel reactor, and then added with 0.04% by weight of a stannous octoate catalyst and 0.05% by weight of diethylene glycol. Under the protection of nitrogen, the temperature of the system was raised to 170° C., and a polymerization reaction was carried out for 9 hours to obtain the reactant A, which had an intrinsic viscosity of 2.03 dl/g as determined at 25° C. in chloroform with a concentration of 0.1 g/dl. The reactant A had a tensile strength of 64 MPa and an elongation at break of 10% through mechanical experiments at 25° C.

(2) preparation of a reactant B: 200 g of a trimethylene carbonate monomer (TMC) was placed in a 3 L stainless steel reactor, and then added with 0.04% by weight of the stannous octoate catalyst and 0.05% by weight of diethylene glycol. Under the protection of nitrogen, the temperature of the system was raised to 180° C., and the polymerization reaction was carried out for 4 hours to obtain the reactant B, which had an intrinsic viscosity of 1.56 dl/g as determined at 25° C. in chloroform with a concentration of 0.1 g/dl. The reactant B had a tensile strength of 4 MPa and an elongation at break of 421% through mechanical experiments at 25° C.

(3) preparation of the PGLT1: 170 g of the reactant A, 150 g of the reactant B and 680 g of glycolide (GA) were added into a 3 L stainless steel reactor, and under the protection of nitrogen, the temperature was raised to 215° C. and maintained for 4 hours to obtain the PGLT1, which had an intrinsic viscosity of 1.45 dl/g as determined at 25° C. in hexafluoroisopropanol with a concentration of 0.1 g/dl.

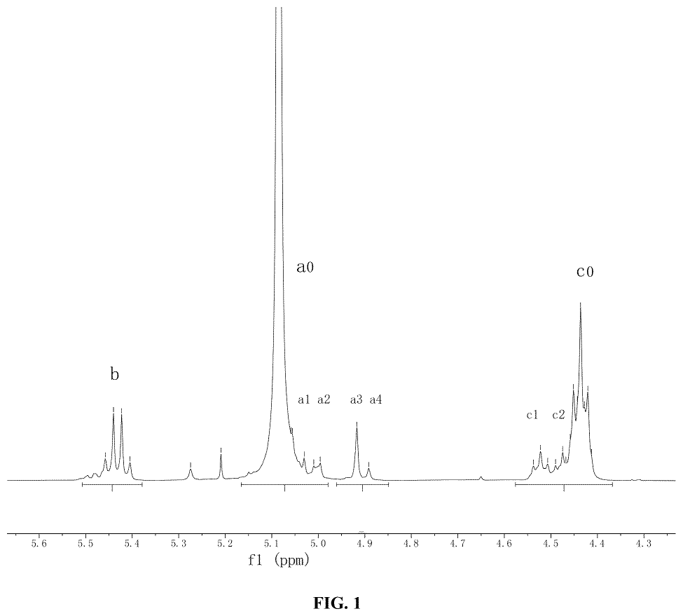

FIG. 1 was a ¹HNMR map of the PGLT copolymer. The weight percentage contents of the glycolide, L-lactide and trimethylene carbonate monomer components in the PGLT1 copolymer were determined by ¹HNMR. With deuterated trifluoroacetic acid as a solvent, the characteristic peak of methylene in the glycolide polymer chain segment appeared at 4.8-5.3 ppm, the characteristic peak of methylene in the lactide polymer segment was between 5.3-5.5 ppm, and the characteristic peak c of o-methylene of an oxygen atom in the trimethylene carbonate polymer chain segment was between 4.3-4.6 ppm. Among them, peak a0 represented the chemical shift of methylene in the homopolymerized chain segment of GA, and peak a1, peak a2, peak a3 and peak a4 respectively represented the chemical shifts of methylene in the random segments formed by chemical reactions such as transesterification of the GA. Among them, peak co represented the chemical shift of o-methylene of an oxygen atom in the homopolymerized chain segment of TMC, and peak c1 and peak c2 respectively represented the chemical shifts of o-methylene of an oxygen atom in the random segments formed by chemical reactions such as TMC transesterification. In view of the above, it could be seen that the numbers of glycolide homopolymerized chain segments, lactide homopolymerized chain segments and trimethylene carbonate homopolymerized chain segments were far more than that of random chain segments, which proved that the PGLT copolymer had been successfully synthesized.

As shown in FIG. 1, the respective proportions could be calculated through the integrated areas of the three peaks A, B and C, and the weight percentage content (Gw) of the glycolide was calculated by the following formula:

$$GW ( \% ) = \frac{116 \times a}{116 \times a + 144 \times 2 \times b + 102 \times c} \times 100$$

$$a = a 0 + a 1 + a 2 + a 3 + a 4 ;$$

By analogy, the weight percentage contents of L-lactide (Lw) and trimethylene carbonate (Tw) were respectively calculated as follows:

$$L W ( \% ) = \frac{144 \times 2 \times b}{116 \times a + 144 \times 2 \times b + 102 \times c} \times 100$$

$$Tw ( \% ) = \frac{102 \times c}{116 \times a + 144 \times 2 \times b + 102 \times c} \times 100$$

$$c = c 0 + c 1 + c 2.$$

The G/L/T proportions (weight %) in the examples and comparative examples calculated by the aforementioned method were shown in Table 1.

The aforementioned PGLT copolymer was injection-molded at about 225° C. to make dumbbell pieces with a thickness of 2 mm, and then stretched at a speed of 10 mm/min on a universal mechanical testing machine to test the tensile yield strength and elongation at break of the material, and stretched at a speed of 1 mm/min. The tensile elastic modulus was the slope of the straight line portion on the stress-strain curve, as shown in Table 1.

The PGLT copolymer synthesized above was tested for crystallinity by differential scanning calorimetry (DSC) at a temperature rising speed of 10° C./min. The crystallinity referred to the crystallinity (C) of the PGA chain segment in the PGLT copolymer. A melting peak of the PGA chain segment not lower than 210° C. would appear in the DSC diagram, and the melting enthalpy value of the melting peak could be calculated. The calculation formula was as follows

$$C ( \% ) = \frac{Δ H}{w \times 203} \times 100$$

where ΔH was a difference (J/g) obtained by subtracting the crystallization enthalpy value (if any) of the PGA chain segment from the melting enthalpy value thereof in the DSC diagram, 203 (J/g) was the melting enthalpy value of the completely crystallized GA (Lebedev B V, Yepstropov A A, Kiparisova V G, Belov B I. Polym Sci USSR 1978;20: 32), was the weight percentage content of the GA, and the results were shown in Table 1.

### Example 2

Preparation of a PGLT copolymer 2 (PGLT2) of glycolide, L-lactide and trimethylene carbonate:

(1) preparation of a reactant A: 160 g of L-lactide (LA) and 40 g of glycolide (GA) were placed in a 3 L of stainless steel reactor, and then added with 0.03% by weight of a stannous octoate catalyst and 0.06% by weight of diethylene glycol. Under the protection of nitrogen, the temperature of the system was raised to 180° C., and a polymerization reaction was carried out for 6 hours to obtain the reactant A, which had an intrinsic viscosity of 1.88 dl/g as determined at 25° C. in chloroform with a concentration of 0.1 g/dl. The reactant A had a tensile strength of 49 MPa and an elongation at break of 10% through mechanical experiments at 25° C.

(2) preparation of a reactant B: 200 g of trimethylene carbonate (TMC) and 40 g of glycolide (GA) were placed in a 3 L stainless steel reactor, and then added with 0.02% by weight of a stannous octoate catalyst and 0.08% by weight of diethylene glycol. Under the protection of nitrogen, the temperature of the system was raised to 170° C., and a polymerization reaction was carried out for 5 hours to obtain the reactant B, which had an intrinsic viscosity of 1.50 dl/g as determined at 25° C. in chloroform with a concentration of 0.1 g/dl. The reactant B had a tensile strength of 4 MPa and an elongation at break of 402% through mechanical experiments at 25° C.

(3) preparation of the PGLT2: 170 g of the reactant A, 150 g of the reactant B and 680 g of glycolide were added into a 3 L stainless steel reactor, and under the protection of nitrogen, the temperature was raised to 220° C. and maintained for 3 hours to obtain the PGLT2, which had an intrinsic viscosity of 1.30 dl/g as determined at 25° C. in hexafluoroisopropanol with a concentration of 0.1 g/dl.

The determination of composition ratio and performance characterization of the PGLT2 were the same as those of Example 1, as shown in Table 1.

### Example 3

The preparation of a PGLT copolymer 3 (PGLT3) of glycolide, L-lactide and trimethylene carbonate included the following steps:

(1) preparation of a reactant A: 180 g of L-lactide (LA) and 20 g of glycolide (GA) were placed in a 3 L stainless steel reactor, and then added with 0.02% by weight of a stannous octoate catalyst and 0.16% by weight of octadecanol. Under the protection of nitrogen, the temperature of the system was raised to 165° C. and a polymerization reaction was carried out for 10 hours to obtain the reactant A, which had an intrinsic viscosity of 2.48 dl/g as determined at 25° C. in chloroform with a concentration of 0.1 g/dl. The reactant A had a tensile strength of 58 MPa and an elongation at break of 10% through mechanical experiments at 25° C.

(2) preparation of a reactant B: 180 g of trimethylene carbonate (TMC) and 20 g of glycolide (GA) were placed in a 3 L stainless steel reactor, and then added with 0.02% by weight of a stannous octoate catalyst and 0.16% by weight of octadecanol. Under the protection of nitrogen, the temperature of the system was raised to 170° C., and a polymerization reaction was carried out for 5 hours to obtain the reactant B, which had an intrinsic viscosity of 1.78 dl/g as determined at 25° C. in chloroform with a concentration of 0.1 g/dl. The reactant B had a tensile strength of 5 MPa and an elongation at break of 455% through mechanical experiments at 25° C.

(3) preparation of the PGLT3: 200 g of the reactant A, 150 g of the reactant B and 650 g of glycolide were added into a 3 L stainless steel reactor, and under the protection of nitrogen, the temperature was raised to 220° C. and maintained for 4 hours to obtain the PGLT3, which had an intrinsic viscosity of 1.62 dl/g as determined at 25° C. in hexafluoroisopropanol with a concentration of 0.1 g/dl.

The determination of composition ratio and performance characterization of the PGLT3 were the same as those of Example 1, as shown in Table 1.

### Example 4

The preparation of a PGLT copolymer 4 (PGLT4) of glycolide, L-lactide and trimethylene carbonate included the following steps:

(1) preparation of a reactant A: 190 g of L-lactide (LA) and 10 g of glycolide (GA) were placed in a 3 L stainless steel reactor, and then added with 0.01% by weight of a stannous octoate catalyst and 0.03% by weight of diethylene glycol. Under the protection of nitrogen, the temperature of the system was raised to 165° C. and a polymerization reaction was carried out for 12 hours to obtain the reactant A, which had an intrinsic viscosity of 2.67 dl/g as determined at 25° C. in chloroform with a concentration of 0.1 g/dl. The reactant A had a tensile strength of 60 MPa and an elongation at break of 7% through mechanical experiments at 25° C.

(2) preparation of a reactant B: 190 g of trimethylene carbonate (TMC) and 10 g of glycolide (GA) were placed in a 3 L stainless steel reactor, and then added with 0.01% by weight of a stannous octoate catalyst and 0.15% by weight of octadecanol. Under the protection of nitrogen, the temperature of the system was raised to 170° C., and a polymerization reaction was carried out for 6 hours to obtain the reactant B, which had an intrinsic viscosity of 1.99 dl/g as determined at 25° C. in chloroform with a concentration of 0.1 g/dl. The reactant B had a tensile strength of 5 MPa and an elongation at break of 460% through mechanical experiments at 25° C.

(3) preparation of the PGLT4: 150 g of the reactant A, 100 g of the reactant B and 750 g of glycolide were added into a 3 L stainless steel reactor, and under the protection of nitrogen, the temperature was raised to 220° C. and maintained for 4 hours to obtain the PGLT4, which had an intrinsic viscosity of 1.73 dl/g as determined at 25° C. in hexafluoroisopropanol with a concentration of 0.1 g/dl.

The determination of composition ratio and performance characterization of the PGLT4 were the same as those of Example 1, as shown in Table 1.

### Example 5

Preparation of a PGLT copolymer 5 (PGLT5) of glycolide, L-lactide and trimethylene carbonate:

(1) preparation of a reactant A: 170 g of L-lactide (LA) and 30 g of glycolide (GA) were placed in a 3 L stainless steel reactor, and then added with 0.01% by weight of a stannous octoate catalyst and 0.13% by weight of dodecanol. Under the protection of nitrogen, the temperature of the system was raised to 165° C. and a polymerization reaction was carried out for 12 hours to obtain the reactant A, which had an intrinsic viscosity of 2.24 dl/g as determined at 25° C. in chloroform with a concentration of 0.1 g/dl. The reactant A had a tensile strength of 48 MPa and an elongation at break of 6% through mechanical experiments at 25° C.

(2) preparation of a reactant B: 170 g of trimethylene carbonate (TMC) and 30 g of glycolide (GA) were placed in a 3 L stainless steel reactor, and then added with 0.01% by weight of a stannous octoate catalyst and 0.13% by weight of dodecanol. Under the protection of nitrogen, the temperature of the system was raised to 170° C., and a polymerization reaction was carried out for 6 hours to obtain the reactant B, which had an intrinsic viscosity of 1.68 dl/g as determined at 25° C. in chloroform with a concentration of 0.1 g/dl. The reactant B had a tensile strength of 5 MPa and an elongation at break of 409% through mechanical experiments at 25° C.

(3) preparation of the PGLT5: 70 g of the reactant A, 130 g of the reactant B and 800 g of glycolide were added into a 3 L stainless steel reactor, and under the protection of nitrogen, the temperature was raised to 205° C. and maintained for 4 hours to obtain the PGLT5, which had an intrinsic viscosity of 1.86 dl/g as determined at 25° C. in hexafluoroisopropanol with a concentration of 0.1 g/dl.

The determination of composition ratio and performance characterization of the PGLT5 were the same as those of Example 1, as shown in Table 1.

### Example 6

Preparation of a PGLT copolymer 6 (PGLT6) of glycolide, DL-lactide and trimethylene carbonate:

(1) preparation of a reactant A: 180 g of DL-lactide (LA) and 20 g of glycolide (GA) were placed in a 3 L stainless steel reactor, and then added with 0.02% by weight of a stannous octoate catalyst and 0.16% by weight of octadecanol. Under the protection of nitrogen, the temperature of the system was raised to 160° C. and a polymerization reaction was carried out for 8 hours to obtain the reactant A, which had an intrinsic viscosity of 2.83 dl/g as determined at 25° C. in chloroform with a concentration of 0.1 g/dl. The reactant A had a tensile strength of 51 MPa and an elongation at break of 11% through mechanical experiments at 25° C.

(2) preparation of a reactant B: it was the same as that in Example 3;

(3) preparation of the PGLT6: 120 g of the reactant A, 180 g of the reactant B and 700 g of glycolide were added into a 3 L stainless steel reactor, and under the protection of nitrogen, the temperature was raised to 220° C. and maintained for 4 hours to obtain the PGLT6, which had an intrinsic viscosity of 1.65 dl/g as determined at 25° C. in hexafluoroisopropanol with a concentration of 0.1 g/dl.

The determination of composition ratio and performance characterization of the PGLT6 were the same as those of Example 1, as shown in Table 1.

### Example 7

The preparation of a PGLT copolymer 7 (PGLT7) of glycolide, L-lactide and trimethylene carbonate included the following steps:

Using the reactant A of Example 1 and the reactant B of Example 4, the reactants were added into a 3 L stainless steel reactor according to a A/B/C mass percentage of 25:13:62, and under the protection of nitrogen, the temperature was raised to 220° C. and maintained for 4 hours, so as to obtain the PGLT7, which had an intrinsic viscosity of 1.57 dl/g as determined at 25° C. in hexafluoroisopropanol with a concentration of 0.1 g/dl.

The determination of composition ratio and performance characterization of the PGLT7 were the same as those of Example 1, as shown in Table 1.

### Example 8

Using the reactant A of Example 1 and the reactant B of Example 4, the reactants were added into a 3 L stainless steel reactor according to a A/B/C mass percentage of 5:20:75, and under the protection of nitrogen, the temperature was raised to 222° C. and maintained for 4 hours, so as to obtain the PGLT8, which had an intrinsic viscosity of 1.53 dl/g as determined at 25° C. in hexafluoroisopropanol with a concentration of 0.1 g/dl.

### Comparative Example 1

A method for preparing a random copolymer 1 (PGLTW1) of glycolide, L-lactide and trimethylene carbonate included the following steps:

720 g of glycolide, 120 g of L-lactide and 160 g of trimethylene carbonate were placed in a 3 L stainless steel reactor, and added with 0.02% of a stannous octoate catalyst and 0.02% by weight of octadecanol. Under the protection of nitrogen, the temperature of the system was raised to 205° C. and a reaction was carried out for 5 hours to obtain the PGLTW1 random copolymer, which had an intrinsic viscosity of 1.76 dl/g as determined at 25° C. in hexafluoroisopropanol with a concentration of 0.1 g/dl.

### Comparative Example 2

A method for preparing a random copolymer 2 (PGLTW2) of glycolide, L-lactide and trimethylene carbonate included the following steps:

800 g of glycolide, 60 g of L-lactide and 140 g of trimethylene carbonate were placed in a 3 L stainless steel reactor, and added with 0.02% of a stannous octoate catalyst and 0.02% by weight of octadecanol. Under the protection of nitrogen, the temperature of the system was raised to 210° C. and a reaction was carried out for 4.5 hours to obtain the PGLTW2 random copolymer, which had an intrinsic viscosity of 1.68 dl/g as determined at 25° C. in hexafluoroisopropanol with a concentration of 0.1 g/dl.

### Comparative Example 3

### Synthesis of PGA:

600 g of glycolide was placed in a 3 L stainless steel reactor, and added with 0.01% of a stannous octoate catalyst and 0.01% by weight of dodecanol. Under the protection of nitrogen, the temperature of the system was raised to 220° C. and a reaction was carried out for 2.5 hours to obtain a PGA homopolymer, which had an intrinsic viscosity of 1.69 dl/g as determined at 25° C. in hexafluoroisopropanol with a concentration of 0.1 g/dl.

### Comparative Example 4

Synthesis of polyL-lactic acid (polyL-lactide PLLA);

The preparation method was the same as that of reactant A in Example 1.

### Comparative Example 5

Synthesis of poly(p-dioxanone) (PPDO):

600 g of a p-dioxanone monomer (PDO) was placed in a 3 L stainless steel reactor, and added with 0.02% of a stannous octoate catalyst and 0.01% by weight of dodecanol. Under the protection of nitrogen, the temperature of the system was raised to 120° C., and a reaction was carried out for 24 hours to obtain a PPDOA homopolymer, which had an intrinsic viscosity of 1.96 dl/g as determined at 25° C. in hexafluoroisopropanol with a concentration of 0.1 g/dl.

### Comparative Example 6

### Preparation of Blended Materials of PGA and PPDO;

420 g of PGA and 80 g of PPDO were physically blended and then injection molded into dumbbell-shaped flaky test samples. The injection temperature was 228° C.

### Comparative Example 7

### Preparation of Blended Materials of PGA and PPDO;

400 g of PGA and 100 g of PPDO were physically blended and then injection molded into dumbbell-shaped flaky test samples. The injection temperature was 228° C.

### Comparative Example 8

### Preparation of Blended Materials of PGA and PTMC;

420 g of PGA and 80 g of PTMC were physically blended and then injection molded into dumbbell-shaped flaky test samples. The injection temperature was 228° C.

### Detection and Characterization:

1. The products obtained in Examples 1-8 and Comparative Examples 1-8 were detected, and the results were shown in Table 1.

TABLE 1
Table of Polymer Performance Results

| | G/L/T | Tensile | Elongation | Melting | | Elastic |
|---|---|---|---|---|---|---|
| | proportion | strength | at break | peak | Crystallinity | modulus |
| Name | (weight %) | (MPa) | (%) | (° C.) | (%) | (MPa) |
| PGLT1 | 66.8/17.3/15.9 | 55 | 70 | 214.2 | 27.3 | 1021 |
| Example 1 | | | | | | |
| PGLT2 | 72.6/14.1/13.3 | 60 | 66 | 215.5 | 35.0 | 1120 |
| Example 2 | | | | | | |
| PGLT3 | 67.5/18.5/14 | 56 | 68 | 215.2 | 28.1 | 1105 |
| Example 3 | | | | | | |
| PGLT4 | 75.9/14.3/9.8 | 71 | 54 | 217.5 | 35.2 | 1202 |
| Example 4 | | | | | | |
| PGLT5 | 82.1/6.3/11.6 | 76 | 56 | 218.3 | 56.6 | 1689 |
| Example 5 | | | | | | |
| PGLT6 | 72.3/11.2/16.5 | 56 | 71 | 215.6 | 34.4 | 963 |
| Example 6 | | | | | | |
| PGLT7 | 61.5/25.6/12.9 | 53 | 62 | 213.4 | 25.8 | 1580 |
| Example 7 | | | | | | |
| PGLT8 | 74.0/5.3/20.7 | 55 | 103 | 216.1 | 39.9 | 806 |
| Example 8 | | | | | | |
| PGLTW1 | 72.5/11.3/16.2 | 42 | 87 | 209.2 | 15.4 | 782 |
| (Comparative | | | | | | |
| Example 1) | | | | | | |
| PGLTW2 | 80.6/5.2/14.2 | 49 | 83 | 212.0 | 19.5 | 968 |
| (Comparative | | | | | | |
| Example 2) | | | | | | |
| PGA | 100/0/0 | 88 | 12 | 222.7 | 83 | 3758 |
| (Comparative | | | | | | |
| Example 3) | | | | | | |
| PLLA | 0/100/0 | 64 | 10 | 176.5 | | 3003 |
| (Comparative | | | | | | |
| Example 4) | | | | | | |
| PPDO | | 27 | 352 | 103.2 | | 995 |
| (Comparative | | | | | | |
| Example 5) | | | | | | |
| Comparative | | 62 | 54 | 221.9 | 68.6 | 1866 |
| Example 6 | | | | | | |
| Comparative | | 56 | 67 | 219.2 | 60.2 | 1235 |
| Example 7 | | | | | | |
| Comparative | | 65 | 59 | 222.6 | 65.0 | 1897 |
| Example 8 | | | | | | |

As could be seen from Table 1, although the content of GA in the absorbable copolymer PGLT prepared by employing Examples 1-8 of the present disclosure was similar to that of the random copolymer prepared in Comparative Example 1, the absorbable copolymer prepared by the present disclosure had higher crystallinity and a higher tensile yield strength up to 76 MPa, which was much higher than that of the poly(p-dioxanone) PPDO in Comparative Example 5. This result further proved the conclusion that the mechanical properties were higher when the crystallinity of the polymer was higher.

The melting peaks of the absorbable copolymers PGLT prepared in Examples 1-7 of the present disclosure were 213-218° C., the melting peaks of the random copolymers of Comparative Example 1 and Comparative Example 2 were lower than 213° C., and the melting peak of the PGA homopolymer of Comparative Example 3 was about 222° C. Therefore, it could be seen that a material with a low crystallization melting peak was more flexible than a material with a high crystallization melting peak, and was easier for thermoplastic processing.

Compared with the PGA homopolymer of Comparative Example 3 and the PLLA homopolymer of Comparative Example 4 that had low elongation at break and lacked toughness, the PGLT random copolymer and the PGLT copolymer prepared by Examples 1-8 of the present disclosure had higher elongation at break and better toughness.

The PPDO in Comparative Example 5 had moderate toughness and degradation time, but its mechanical strength was low (with tensile yield strength<30 MPa). Blending of PGA and soft materials was conducted in each of Comparative Examples 6, 7 and 8, and thus the tensile strength and toughness could meet the application requirements.

It could be seen from Examples 1-8 that, the proportions of the reactant A, the reactant B and the reactant C were different, and the mechanical strength and degradation times of the copolymers were different. The mechanical strength was higher when the content of LA (the reactant A) and GA (the reactant C) polymerized chain segments in the hard segment was higher, and the contribution of the GA polymerized chain segment to the mechanical strength was greater than that of the LA chain segment. Therefore, it could be seen that when the polymer was formed by LLA instead of GA, or the polymer was formed when the content of LLA was greater than that of GA, although still containing TMC polymer chain segments, the formed PGLT copolymer was easily subjected to stress-relaxation under stress, producing permanent deformation.

2. The polymers obtained in Examples 1-8 and Comparative Examples 1-8 were tested for mechanical properties of degradation in vitro.

Test method: It was carried out in a phosphate buffer solution at 37° C., and the shape of the sample was dumbbell-shaped and flaky. The method was consistent with the test method of Example 1. The samples were taken out at different time points for testing, and the results were shown in Table 2.

TABLE 2
Mechanical results of polymer degradation in vitro

| | Tensile yield strength (MPa) | | | | | | |
|---|---|---|---|---|---|---|---|
| | 0 | 7 | 10 | 14 | 18 | 21 | 25 |
| Material | day | days | days | days | days | days | days |
| PGLT1 Example 1 | 55 | 45 | 33 | 22 | 15 | 10 | 3 |
| PGLT2 Example 2 | 60 | 37 | 27 | 19 | 9 | 3 | 0 |
| PGLT3 Example 3 | 56 | 38 | 29 | 22 | 17 | 9 | 4 |
| PGLT4 Example 4 | 71 | 43 | 33 | 15 | 12 | 8 | 3 |
| PGLT5 Example 5 | 76 | 59 | 35 | 23 | 13 | 8 | 0 |
| PGLT6 Example 6 | 56 | 48 | 30 | 29 | 18 | 13 | 6 |
| PGLT7 Example 7 | 58 | 46 | 38 | 24 | 14 | 9 | 5 |
| PGLT8 Example 8 | 55 | 47 | 37 | 28 | 18 | 12 | 4 |
| Comparative | 42 | 16 | 7 | 0 | | | |
| Example 1 | | | | | | | |
| Comparative | 49 | 20 | 9 | 2 | 0 | | |
| Example 2 | | | | | | | |
| Comparative | 88 | 52 | 28 | 19 | 0 | | |
| Example 3 | | | | | | | |
| Comparative | 64 | 68 | 67 | 68 | 66 | 65 | 67 |
| Example 4 | | | | | | | |
| Comparative | 27 | 27 | 24 | 21 | 17 | 12 | 9 |
| Example 5 | | | | | | | |
| Comparative | 62 | 12 | 3 | 0 | | | |
| Example 6 | | | | | | | |
| Comparative | 56 | 14 | 5 | 0 | | | |
| Example 7 | | | | | | | |
| Comparative | 65 | 41 | 18 | 5 | 0 | | |
| Example 8 | | | | | | | |

As could be seen from Table 2, the tensile strength maintenance times of the PGLT copolymers of Examples 1-8 obtained by the preparation method of the present disclosure were obviously longer than those of the PGLT random copolymers formed in Comparative Example 1 and Comparative Example 2, which was mainly because the disordered arrangement of monomers of the random copolymers reduced the crystallization of PGA chain segments and increased amorphous components that degraded rapidly. Although the degradation times of PGA in Comparative Example 3 and PLLA in Comparative Example 4 were longer than those of the random copolymers, the disadvantage was that the material lacked toughness. For the PPDO in Comparative Example 5, the material had an enough degradation strength maintenance time, but its strength and hardness were insufficient, so it was not suitable for preparing absorbable closure clips. The strength maintenance times of the materials of Comparative Examples 6 and 7 both did not exceed 10 days. The material of Comparative Example 8 was a composite material of PGA blended with PTMC. Although the degradation speed of the PTMC was much slower than that of the PGA, the degradation strength maintenance time was not significantly improved compared with that of the PGA.

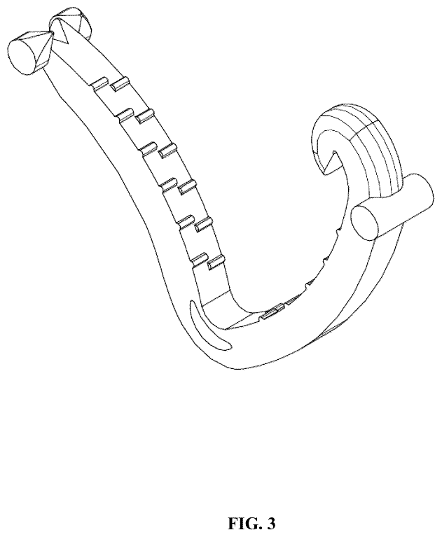

3. The materials of Examples 1-8 and Comparative Examples 1-8 and polyoxymethylene were sequentially injection molded to make absorbable tissue closure clips, and then subjected to performance tests: Clamping force and degradation maintenance time. The results were shown in Table 3. The structure of the closure clip was shown in FIG. 3. The temperatures of the head and nozzle of the injection molding machine were 190° C.-230° C., the injection molding pressure was 70-150 MPa, and the injection molding speed was greater than 100 mm/s. In the field of medical instruments, the clamping force of the absorbable closure clip should be greater than 5 N.

Test method of clamping force: Two non-woven fabrics with a width of 5 mm±1 mm were taken, stacked in the middle of the closure clip at a clamping position, fixed on a mechanical tester, and then stretched at a speed of 10 mm/min until the closure clip was pulled off or broken, and the tension value at this time was recorded.

### Table 3 Results of Clamping Forces of Closure Clips

As could be seen from Table 3, the maintenance times of the degradation of the clamping forces of the absorbable closure clips prepared by employing the PGLT copolymers prepared in Examples 1-8 of the present disclosure could be at least 14 days. Compared with the closure clips supported by random copolymers of Comparative Example 1 and Comparative Example 2, the in vitro degradation time of the closure clip of the random copolymer was less than 10 days, which is much lower than that of the material prepared by the present disclosure. The degradation times of PGA composite materials prepared by blending in Comparative Examples 6, 7 and 8 were less than 10 days. In Comparative Example 5, the clamping force of the closure clip made of the PPDO material could only reach 15-16 N in the initial period. If the clamping force was too low, the closure clip was easy to slip and displace in use and cannot penetrate fascia tissues. The materials of the PGA in Comparative Example 3 and the PLLA in Comparative Example 4 were hard, so the prepared closure clips cannot be closed, and they were easy to break when closed.

In view of the above, it could be seen that the PGLT copolymer prepared by the present disclosure has high strength and good toughness, and a degradation time that can reach 14 days or more. Considering the mechanical properties and degradation time of the material, the PGLT copolymer is suitable for preparing absorbable tissue closure clips, soft tissue nails, suture clips, suture anchors, surgical sutures, puncture occluders or anal fistula occludes, etc.

What is claimed is:

1. A method for preparing an absorbable copolymer, comprising: forming a PGLT copolymer by stepwise copolymerization of glycolide, lactide and trimethylene carbonate, wherein in the PGLT copolymer, a weight percentage of the glycolide is 60%-85%, a weight percentage of the lactide is 5%-25%, and a weight percentage of the trimethylene carbonate is 8%-25%, and in a process of stepwise copolymerization, the lactide and the trimethylene carbonate are firstly polymerized to form a prepolymer and then the prepolymer is copolymerized with the glycolide, and the PGLT copolymer contains three chain segments of polymerized glycolide, polymerized lactide and polymerized trimethylene carbonate, specifically comprising the following steps:
(1) a reactant A: forming a homopolymer or copolymer by polymerization of the lactide or of the lactide and the glycolide in the presence of an initiator and a catalyst;
(2) a reactant B: forming a homopolymer or copolymer by polymerization of the trimethylene carbonate or of the trimethylene carbonate and the glycolide in the presence of an initiator and a catalyst; and
(3) copolymerization: using the glycolide as a reactant C, melting and blending the reactant C, the reactant B and the reactant A evenly, and then polymerizing in an air-insulated reactor to form the PGLT copolymer.

2. The method for preparing an absorbable copolymer according to claim 1, comprising:
(1) a reactant A: adding 0.01%-0.2% by weight of the initiator and 0.01%-0.1% by weight of the catalyst, respectively adding the lactide and the glycolide into a reactor under the protection of nitrogen or a vacuum condition, heating to 150° C.-180°C., maintaining at the reaction temperature for 6-15 hours, and conducting ring-opening polymerization to obtain the reactant A;
(2) a reactant B: adding 0.01%-0.2% by weight of the initiator and 0.01%-0.1% by weight of the catalyst, respectively adding the trimethylene carbonate and the glycolide into a reactor under the protection of nitrogen or a vacuum condition, heating to 150° C.-180° C., maintaining at the reaction temperature for 2-8 hours, and conducting ring-opening polymerization to obtain the reactant B;
(3) copolymerization: using the glycolide as the reactant C, melting and blending the reactant C, the reactant A and the reactant B evenly, and polymerizing at 200° C.-230° C. in an air-insulated reactor to form the PGLT copolymer containing the glycolide, the lactide and the trimethylene carbonate.

3. The method for preparing an absorbable copolymer according to claim 1 or 2, wherein: the initiator is a monohydric, dihydric or polyhydric alcohol among fatty alcohols, and the catalyst is stannous octoate.

4. The method for preparing an absorbable copolymer according to claim 1 or 2, wherein: the lactide is one or more of L-lactide, DL-lactide or D-lactide.

5. The method for preparing an absorbable copolymer according to claim 1 or 2, wherein:
in the reactant A, the weight percentage of the glycolide is 0-20%, and in the reactant B, the weight percentage of the glycolide is 0-20%.

6. An absorbable copolymer, wherein: the PGLT copolymer has an elastic modulus of 600 MPa-2,300 MPa, a tensile yield strength of greater than 40 MPa, and a tensile elongation at break of no less than 40%.

7. The absorbable copolymer according to claim 6, wherein: the PGLT copolymer has an elastic modulus of 800 MPa-2,000 MPa, a tensile yield strength of greater than 50 MPa, and a tensile elongation at break of no less than 50%.

8. The absorbable copolymer according to claim 6 or 7, wherein: the PGLT copolymer has an intrinsic viscosity of 0.8-2.5 dl/g determined at 25° C. in hexafluoroisopropanol with a concentration of 0.1 g/dl.

9. The absorbable copolymer according to claim 6 or 7, wherein: the tensile strength of the PGLT copolymer is no less than 5 MPa after being soaked in a phosphate buffer solution at 37° C. for 14 days.

10. Use of an absorbable copolymer, wherein: the absorbable copolymer can be used for preparing tissue closure clips, suture clips, suture anchors, anastomosis nails, surgical sutures, puncture occluders or anal fistula occludes.
